(19) United States
(12) Patent Application Publication
(10) Pub. No.: US 2017/0112940 A1
(43) Pub. Date: Apr. 27, 2017

(54) Modified Collagen Hybridizing Peptides And Uses Thereof

(71) Applicant: University of Utah Research Foundation, Salt Lake City, UT (US)

(72) Inventors: S. Michael Yu, Salt Lake City, UT (US); Yang Li, Holladay, UT (US); Hendra Wahyudi, Salt Lake City, UT (US)

(21) Appl. No.: 15/331,348

(22) Filed: Oct. 21, 2016

Related U.S. Application Data
(60) Provisional application No. 62/285,258, filed on Oct. 23, 2015.
(60) Provisional application No. 62/310,937, filed on Mar. 21, 2016.

(51) Int. Cl. A61K 51/00

(52) U.S. Cl. CPC A61K 47/48238

(57) ABSTRACT

Disclosed are peptide conjugates comprising an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ², wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine. Disclosed are peptide conjugates comprising an active agent, a spacer moiety, and a peptide, wherein the peptide comprises the sequence (X₁₋Y₁₋Z₁)-(X₂₋Y₂₋Z₂)-(Xₙ₋Yₙ₋Zₙ) (SEQ ID NO:1-39, 82), wherein n=1-41, and X, Y, and Z are glycine, proline, a modified proline and/or aza-glycine, and at least one of the amino acids in the sequence is aza-glycine. Also disclosed are compositions and nanoparticles comprising these peptide conjugates. Also disclosed are methods of detecting and methods of treating using the peptide conjugates or compositions comprising the peptide conjugates.

### CROSS-REFERENCE TO RELATED APPLICATIONS

This application claims benefit of U.S. Provisional Application No. 62/285,258, filed Oct. 23, 2015 and U.S. Provisional Application No. 62/310,937, filed Mar. 21, 2016, both of which are hereby incorporated herein by reference in their entirety.

### STATEMENT REGARDING FEDERALLY SPONSORED RESEARCH

This invention was made with government support under grants AR060484 and AR065124 awarded by the National Institutes of Health and grant W81XWH-12-1-0555 awarded by the Department of Defense. The government has certain rights in the invention.

### REFERENCE TO SEQUENCE LISTING

The Sequence Listing submitted Oct. 21, 2016 as a text file named “21101_0320U3_Sequence_Listing.txt,” created on Oct. 21, 2016, and having a size of 148,266 bytes is hereby incorporated by reference pursuant to 37 C.F.R. §1.52(e)(5).

### BACKGROUND

Collagen is a major structure protein found in almost all human tissue. Degraded collagen is present in damaged tissues and is highly associated with many critical human diseases and injuries. Collagen hybridizing peptides (CHP) are capable of specifically binding to the degraded collagen but not the intact collagen molecule. The CHP binds to the degraded collagen by forming a collagen-like triple helical structure. Collagen hybridizing peptide sequences containing Aza-Glycine residues can significantly increase the stability of the collagen triple helix structure by forming additional hydrogen bonds. The aza-Gly containing CHP will not bind to collagen but can bind to degraded collagen with high affinity.

What is needed are compositions and methods for detecting degraded collagen as well as methods of targeting active agents to degraded collagen.

### BRIEF SUMMARY

Disclosed are peptide conjugates comprising an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine.

Disclosed are compositions comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine.

Disclosed are nanoparticles comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine.

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine.

Disclosed are kits comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the melting temperature of Ac(GPO)₃AzGPO(GPO)₃ Ac(GPO)₂AzGPOGPOAzGPO(GPO)₂ in 1× PBS. The sample concentration tested is 200 uM. 7 is Ac-GPO-GPO-GPO-GPO-GPO-GPO-GPO (This is positive control). 7-1azGly is Ac-GPO-GPO-GPO-azGlyPO-GPO-GPO-GPO. 7-2azGly is Ac-GPO-GPO-azGlyPO-GPO-azGlyPO-GPO-GPO. Melting Temperature of 7 is 54° C. Melting Temperature of 7-1azGly is 65° C. Melting Temperature of 7-2azGly is 77° C.

FIG. 2 shows a synthetic scheme of Ac-(GPO)3-azGlyPO-(GPO)3.

FIG. 3 shows gelatin binding at certain time points (Raw). 10: 10 μM added to crosslinked gelatin gel. The bar graph shows the amount of peptide remaining on the gelatin film after peptide binding and washing (all at 37° C.) for 1 day. 7 is CF-GGG-GPO-GPO-GPO-GPO-GPO-GPO-GPO (positive control). 7-1azGly is CF-GGG-GPO-GPO-GPO-azGlyPO-GPO-GPO-GPO. 7-2azGly is CF-GGG-GPO-GPO-azGlyPO-GPO-azGlyPO-GPO-GPO. Random9 is Scramble sequence of CF-GGG-(GPO)9 The sequence is CF-GGG-PGO-GPG-POP-OGO-GOP-PGO-OPG-GOO-PPG. (negative control). At 37 C, 7-1azGly has 1.6-fold higher binding affinity than 7 to gelatin.

FIGS. 4A and 4B show A) Fluorescent image of SDS-PAGE stained with 35 μM of CFG₃(GPO)₃AzGPO(GPO)₃; B) SDS-PAGE stained with Commassie Blue. Lane 1: MW marker, Lane 2: 50 ug BSA, Lane 3: 5 ug Denatured Collagen (col), Lane 4: 10 ug Denatured Collagen (col). Commasie blue (4B) stains collagen (Lanes 3 and 4) as well as BSA (lane 2). In contrast, the aza-CHP (4A) stains only collagen (lanes 3 and 4). There is almost no signal from lane 2.

FIG. 5 shows a synthetic scheme of CF-GGG-(GPO)3-azGlyPO-(GPO)3.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide conjugate is disclosed and discussed and a number of modifications that can be made to a number of molecules including the peptide conjugate are discussed, each and every combination and permutation of the peptide conjugate and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

The term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a disease or injury involving collagen damage can refer to reducing or eliminating the amount of damaged/denatured collagen. Treatment can also be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The term “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient.”

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Peptides

Disclosed are peptides referred to as collagen hybridizing peptides. In some instances, the peptides comprise the sequence (Gly-Pro-Hyp)n¹-azGly-Pro-Hyp-(Gly-Pro-Hyp)n² (SEQ ID NOs:40-78), wherein n¹ and n² are each an integer from 1 to 20. In some instances, n¹ and n² are each 3.

Disclosed are peptides comprising the sequence (Gly-Pro-Hyp)₃-azGly-Pro-Hyp-(Gly-Pro-Hyp)₃ (SEQ ID NO:79); (Pro-Hyp-Gly)₃-Pro-Hyp-azGly-(Pro-Hyp-Gly)₃ (SEQ ID NO:80); or (Pro-Hyp-Gly)₃-Pro-Pro-azGly-(Pro-Hyp-Gly)₃ (SEQ ID NO:81).

Disclosed are peptides comprising the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least two of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20.

Disclosed are peptides comprising the sequence (X₁₋Y₁₋Z₁)-(X₂₋Y₂₋Z₂)-(Xₙ₋Yₙ₋Zₙ) (SEQ ID NO:1-39, 82), wherein n can be from 1-41, and X, Y, and Z are glycine, proline, a modified proline or aza-glycine, and at least one of the amino acids in the sequence is aza-glycine. Disclosed are peptides that comprise between 1 and 41 amino acid triplets of the sequence X-Y-Z, wherein X, Y, and Z can be glycine, proline, a modified proline or aza-glycine and each triplet can have the same or different sequence. In some instances, the X, Y, and Z in each triplet can be the same as the X, Y, and Z in every triplet, respectively. In some instances, the X, Y, and Z in each triplet can be different from one or more of the X, Y, and Z's, respectively, in the other triplets.

In some instances, peptides with the sequence (X₁₋Y₁₋Z₁)-(X₂₋Y₂₋Z₂)-(Xₙ₋Yₙ₋Zₙ) can include, but are not limited to AzGPO-GPO-AzGPO-GPO-GPO-GPO-GPO (SEQ ID NO:83); AzGPO-GPO-GPO-AzGPO-GPO-GPO-GPO (SEQ ID NO:84); GPO-AzGPO-AzGPO-GPO-GPO-GPO-GPO (SEQ ID NO:85); GPO-AzGPO-GPO-AzGPO-GPO-GPO-GPO (SEQ ID NO:86); GPO-GPO-AzGPO-GPO-AzGPO-GPO-GPO (SEQ ID NO:87); GPO-AzGPO-AzGPO-AzGPO-GPO-GPO-GPO (SEQ ID NO:88); AzGPO-GPO-AzGPO-AzGPO-GPO-GPO-GPO (SEQ ID NO:89); AzGPO-GPO-AzGPO-GPO-AzGPO-GPO-GPO (SEQ ID NO:90); AzGPO-GPO-AzGPO-GPO-GPO-AzGPO-GPO (SEQ ID NO:91); GPO-AzGPO-GPO-AzGPO-GPO-AzGPO-GPO (SEQ ID NO:92); AzGPO-GPO-AzGPO-AzGPO-AzGPO-GPO-GPO (SEQ ID NO:93); AzGPO-GPO-GPO-AzGPO-AzGPO-AzGPO-GPO (SEQ ID NO:94); AzGPO-GPO-AzGPO-GPO-AzGPO-AzGPO-GPO (SEQ ID NO:95); AzGPO-GPO-AzGPO-GPO-AzGPO-GPO-AzGPO (SEQ ID NO:96).

### C. Peptide Conjugates

Disclosed are peptide conjugates comprising an active agent, a spacer moiety, and a peptide, wherein the peptide can be any of the disclosed peptides.

Disclosed are peptide conjugates comprising an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20.

Disclosed are peptide conjugates comprising an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein no more than one of Xaa₁, Xaa₂, Xaa₃, Xaa₄, Xaa₅, Xaa₆, Xaa₇, Xaa₈, or Xaa₉ is aza-glycine. In some instances, Xaa₁, Xaa₂, and Xaa₃ are not the same amino acid. In some instances, Xaa₄, Xaa₅, and Xaa₆ are not the same amino acid. In some instances, Xaa₇, Xaa₈, and Xaa₉ are not the same amino acid. In some instances, at least two of Xaa₁, Xaa₂, and Xaa₃ are not the same amino acid. In some instances, at least two of Xaa₄, Xaa₅, and Xaa₆ are not the same amino acid. In some instances, at least two of Xaa₇, Xaa₈, and Xaa₉ are not the same amino acid.

Disclosed are peptide conjugates comprising an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein the peptides comprises the sequence (Gly-Pro-Hyp)₃-azGly-Pro-Hyp-(Gly-Pro-Hyp)₃ (SEQ ID NO:79); (Pro-Hyp-Gly)₃-Pro-Hyp-azGly-(Pro-Hyp-Gly)₃ (SEQ ID NO:80); or (Pro-Hyp-Gly)₃-Pro-Pro-azGly-(Pro-Hyp-Gly)₃ (SEQ ID NO:81).

Disclosed are peptide conjugates comprising an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the active agent can be a detectable moiety or a therapeutic agent. In some instances, wherein the detectable moiety is a fluorescent dye, radioactive isotope, magnetic bead, metallic bead, colloidal particle, near-infra red dye, contrast agents (e.g. MRI contrast agents such as but not limited to, Gadolinium complex, ferric ammonium citrate, and mangafodipar trisodium), or an electron-dense reagents. In some instances, the detectable moiety can be attached to the N-terminal portion of the peptide. Thus, detectable moieties can be, but are not limited to, fluorescent moieties, radioactive moieties, electronic moieties, and indirect moieties such as biotin or digoxigenin. When indirect moieties are used, a secondary binding agent that binds the indirect moiety can be used to detect the presence of a bound collagen hybridizing peptide. These secondary binding agents can comprise antibodies, haptens, or other binding partners (e.g., avidin) that bind to the indirect moieties. In some instances, a detectable moiety can be attached to the N-terminal, C-terminal, or both portions of the peptide. In some instances, the therapeutic agent can be a therapeutic known to treat a disease or injury involving collagen damage. For example, the therapeutic agent can be, but is not limited to, any suitable pharmaceutical or other therapeutic agent, including but not limited to, osteogenic promoters, antimicrobials, anti-inflammatory agents, polypeptides such as recombinant proteins, cytokines or antibodies, small molecule chemicals or any combination thereof. In some instances, a therapeutic agent can be a cancer drug, arthritis drug or osteoporosis drug. Therapeutic agents can be capable of promoting bone growth, decreasing inflammation, promoting collagen stability. Examples of therapeutic agents can include, but is not limited to, bone morphogenic protein (BMP), G-CSF, FGF, BMP-2, BMP-3, FGF-2, FGF-4, anti-sclerostin antibody, growth hormone, IGF-1, VEGF, TGF-β, KGF, FGF-10, TGF-α, TGF-β1, TGF-β receptor, CT, GH, GM-CSF, EGF, PDGF, celiprolol, activins, connective tissue growth factors, MMP inhibitors, cathepsin inhibitors, interleukin inhibitors, TRAIL, VEGF inhibitors, and CD binding agents. In some instances, a therapeutic agent can be an antibody such as, but not limited to, Avastin, Eylea, Humira, ReoPro, Campath, tocilizumab, Ilaris, Removab, Cimzia, Erbitux, Zenapax, Prolia, Raptiva, Rexomun, Abegrin, HuZAF, Simponi, Igovomab, IMAB362, Imciromab, Remicade, Yervoy, Tysabri, Theracim, OvaRex, Vectibix, Theragyn, Omnitarg, Cyramza, Lucentis, Antova, Actemra, Herceptin, Ektomab, Stelara, HumaSPECT, HuMax-EGFr, HuMax-CD4. A therapeutic agent can target tumors, arthritis, or osteoporosis.

In some instances, a disease or injury involving collagen damage can be, but is not limited to, cartilage/bone injury, tendon/ligament injury, corneal injury, and disease with high collagen remodeling activity such as cancer, arthritis, osteoporosis, fibrosis, and vulnerable plaques.

Disclosed are peptide conjugates comprising an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the spacer moiety can be located between the active agent and the peptide. In some instances, the spacer moiety can comprise aminohexanoic acid. In some instances, the spacer moiety can be one or more glycines or PEG.

Disclosed are peptide conjugates comprising an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the peptides can have a higher affinity to degraded collagen than a conventional collagen hybridizing peptide. In some instances, the peptides do not bind native collagen.

Disclosed are peptide conjugates comprising an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the N-terminal amino acid comprises an acetyl group. In some instances, the C-terminal amino acid comprises an amino group.

Disclosed are peptide conjugates comprising an active agent, a spacer moiety, and a peptide, wherein the peptide comprises the sequence (X₁₋Y₁₋Z₁)-(X₂₋Y₂₋Z₂)-(Xₙ₋Yₙ₋Zₙ) (SEQ ID NO:1-39, 82), wherein n=1-41, and X, Y, and Z are glycine, proline, a modified proline and/or aza-glycine, and at least one of the amino acids in the sequence is aza-glycine. Disclosed are peptides that comprise between 1 and 41 amino acid triplets of the sequence X-Y-Z, wherein X, Y, and Z can be glycine, proline, a modified proline or aza-glycine each triplet can have the same or different sequence. In some instances, the X, Y, and Z in each triplet can be the same as the X, Y, and Z in every triplet, respectively. In some instances, the X, Y, and Z in each triplet can be different from one or more of the X, Y, and Z′s, respectively, in the other triplets.

### D. Compositions

Disclosed are compositions comprising one or more of the peptides or peptide conjugates disclosed herein.

Disclosed are compositions comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20.

Disclosed are compositions comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein no more than one of Xaa₁, Xaa₂, Xaa₃, Xaa₄, Xaa₅, Xaa₆, Xaa₇, Xaa₈, or Xaa₉ is aza-glycine. In some instances, Xaa₁, Xaa₂, Xaa₃ are not the same amino acid. In some instances, Xaa₄, Xaa₅, and Xaa₆ are not the same amino acid. In some instances, Xaa₇, Xaa₈, and Xaa₉ are not the same amino acid. In some instances, at least two of Xaa₁, Xaa₂, and Xaa₃ are not the same amino acid. In some instances, at least two of Xaa₄, Xaa₅, and Xaa₆ are not the same amino acid. In some instances, at least two of Xaa₇, Xaa₈, and Xaa₉ are not the same amino acid.

Disclosed are compositions comprising a peptide conjugate, wherein the peptide conjugate comprises an active agent, a spacer moiety, and a peptide, wherein the peptide comprises the sequence ((Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein the peptides comprises the sequence (Gly-Pro-Hyp)₃-azGly-Pro-Hyp-(Gly-Pro-Hyp)₃ (SEQ ID NO:79); (Pro-Hyp-Gly)₃-Pro-Hyp-azGly-(Pro-Hyp-Gly)₃ (SEQ ID NO:80); or (Pro-Hyp-Gly)₃-Pro-Pro-azGly-(Pro-Hyp-Gly)₃ (SEQ ID NO:81).

Disclosed are compositions comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the active agent can be a detectable moiety or a therapeutic agent. In some instances, wherein the detectable moiety is a fluorescent dye, radioactive isotope, magnetic bead, metallic bead, colloidal particle, near-infra red dye, or an electron-dense reagents. In some instances, the detectable moiety can be attached to the N-terminal portion of the peptide. Thus, detectable moieties can be, but are not limited to, fluorescent moieties, radioactive moieties, electronic moieties, and indirect moieties such as biotin or digoxigenin. When indirect moieties are used, a secondary binding agent that binds the indirect moiety can be used to detect the presence of a bound collagen hybridizing peptide. These secondary binding agents can comprise antibodies, haptens, or other binding partners (e.g., avidin) that bind to the indirect moieties. In some instances, a detectable moiety can be attached to the N-terminal, C-terminal, or both portions of the peptide. In some instances, the therapeutic agent can be a therapeutic known to treat a disease or injury involving collagen damage. For example, the therapeutic agent can be, but is not limited to, any suitable pharmaceutical or other therapeutic agent, including but not limited to, osteogenic promoters, antimicrobials, anti-inflammatory agents, polypeptides such as recombinant proteins, cytokines or antibodies, small molecule chemicals or any combination thereof. In some instances, a therapeutic agent can be a cancer drug, arthritis drug or osteoporosis drug. Therapeutic agents can be capable of promoting bone growth, decreasing inflammation, promoting collagen stability. Examples of therapeutic agents can include, but is not limited to, bone morphogenic protein (BMP), G-CSF, FGF, BMP-2, BMP-3, FGF-2, FGF-4, anti-sclerostin antibody, growth hormone, IGF-1, VEGF, TGF-β, KGF, FGF-10, TGF-α, TGF-β1, TGF-β receptor, CT, GH, GM-CSF, EGF, PDGF, celiprolol, activins, connective tissue growth factors, MMP inhibitors, cathepsin inhibitors, interleukin inhibitors, TRAIL, VEGF inhibitors, or CD binding agents. In some instances, a therapeutic agent can be an antibody such as, but not limited to, Avastin, Eylea, Humira, ReoPro, Campath, tocilizumab, Ilaris, Removab, Cimzia, Erbitux, Zenapax, Prolia, Raptiva, Rexomun, Abegrin, HuZAF, Simponi, Igovomab, IMAB362, Imciromab, Remicade, Yervoy, Tysabri, Theracim, OvaRex, Vectibix, Theragyn, Omnitarg, Cyramza, Lucentis, Antova, Actemra, Herceptin, Ektomab, Stelara, HumaSPECT, HuMax-EGFr, HuMax-CD4. A therapeutic agent can target tumors, arthritis, or osteoporosis.

Disclosed are compositions comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the spacer moiety can be located between the active agent and the peptide. In some instances, the spacer moiety can comprise aminohexanoic acid. In some instances, the spacer moiety can be one or more glycines or PEG.

Disclosed are compositions comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the peptides can have a higher affinity to degraded collagen than a conventional collagen hybridizing peptide. In some instances, the peptides do not bind native collagen.

Disclosed are compositions comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the N-terminal amino acid comprises an acetyl group. In some instances, the C-terminal amino acid comprises an amino group.

Disclosed are compositions comprising a peptide conjugate, wherein the peptide conjugate comprises an active agent, a spacer moiety, and a peptide, wherein the peptide comprises the sequence (X₁-Y₁-Z₁)-(X₂-Y₂-Z₂)-(Xn-Yn-Zn) (SEQ ID NOs:1-39, 82), wherein n can be from 1-41, and X, Y, and Z are glycine, proline, a modified proline and/or aza-glycine, and at least one of the amino acids in the sequence is aza-glycine. Disclosed are peptides that comprise between 1 and 41 amino acid triplets of the sequence X-Y-Z, wherein X, Y, and Z can be glycine, proline, a modified proline or aza-glycine and each triplet can have the same or different sequence. In some instances, the X, Y, and Z in each triplet can be the same as the X, Y, and Z in every triplet, respectively. In some instances, the X, Y, and Z in each triplet can be different from one or more of the X, Y, and Z′s, respectively, in the other triplets. Also disclosed herein are any of the disclosed compositions further comprising a pharmaceutically acceptable carrier. For example, disclosed are compositions comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, further comprising a pharmaceutically acceptable carrier.

By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, or conjugate of the invention is not compromised. Pharmaceutical compositions may also include one or more active ingredients (in addition to the composition of the invention) such as antimicrobial agents, anti-inflammatory agents, anesthetics, and the like.

### E. Nanoparticles

Disclosed are nanoparticles comprising one or more of the disclosed peptides or peptide conjugates.

Disclosed are nanoparticles comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20.

Disclosed are nanoparticles comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein no more than one of Xaa₁, Xaa₂, Xaa₃, Xaa₄, Xaa₅, Xaa₆, Xaa₇, Xaa₈, or Xaa₉ is aza-glycine. In some instances, Xaa₁, Xaa₂, and Xaa₃ are not the same amino acid. In some instances, Xaa₄, Xaa₅, and Xaa₆ are not the same amino acid. In some instances, Xaa₇, Xaa₈, and Xaa₉ are not the same amino acid. In some instances, at least two of Xaa₁, Xaa₂, and Xaa₃ are not the same amino acid. In some instances, at least two of Xaa₄, Xaa₅, and Xaa₆ are not the same amino acid. In some instances, at least two of Xaa₇, Xaa₈, and Xaa₉ are not the same amino acid.

Disclosed are nanoparticles comprising a peptide conjugate, wherein the peptide conjugate comprises an active agent, a spacer moiety, and a peptide, wherein the peptide comprises the sequence (X1-Y1-Z1)-(X2-Y2-Z2)-(Xn-Yn-Zn) (SEQ ID NOs:1-39, 82), wherein n can be from 1-41, and X, Y, and Z are glycine, proline, a modified proline and/or aza-glycine, and at least one of the amino acids in the sequence is aza-glycine.

Disclosed are nanoparticles comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein the peptides comprises the sequence (Gly-Pro-Hyp)₃-azGly-Pro-Hyp-(Gly-Pro-Hyp)₃ (SEQ ID NO:79); (Pro-Hyp-Gly)₃-Pro-Hyp-azGly-(Pro-Hyp-Gly)₃ (SEQ ID NO:80); or (Pro-Hyp-Gly)₃-Pro-Pro-azGly-(Pro-Hyp-Gly)₃ (SEQ ID NO:81).

Disclosed are nanoparticles comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the active agent can be a detectable moiety or a therapeutic agent. In some instances, wherein the detectable moiety is a fluorescent dye, radioactive isotope, magnetic bead, metallic bead, colloidal particle, near-infra red dye, or an electron-dense reagents. In some instances, the detectable moiety can be attached to the N-terminal portion of the peptide. Thus, detectable moieties can be, but are not limited to, fluorescent moieties, radioactive moieties, electronic moieties, and indirect moieties such as biotin or digoxigenin. When indirect moieties are used, a secondary binding agent that binds the indirect moiety can be used to detect the presence of a bound collagen hybridizing peptide. These secondary binding agents can comprise antibodies, haptens, or other binding partners (e.g., avidin) that bind to the indirect moieties. In some instances, a detectable moiety can be attached to the N-terminal, C-terminal, or both portions of the peptide. In some instances, the therapeutic agent can be a therapeutic known to treat a disease or injury involving collagen damage. For example, the therapeutic agent can be, but is not limited to, any suitable pharmaceutical or other therapeutic agent, including but not limited to, osteogenic promoters, antimicrobials, anti-inflammatory agents, polypeptides such as recombinant proteins, cytokines or antibodies, small molecule chemicals or any combination thereof. In some instances, a therapeutic agent can be a cancer drug, arthritis drug or osteoporosis drug. Therapeutic agents can be capable of promoting bone growth, decreasing inflammation, promoting collagen stability. Examples of therapeutic agents can include, but is not limited to, bone morphogenic protein (BMP), G-CSF, FGF, BMP-2, BMP-3, FGF-2, FGF-4, anti-sclerostin antibody, growth hormone, IGF-1, VEGF, TGF-β, KGF, FGF-10, TGF-α, TGF-β1, TGF-β receptor, CT, GH, GM-CSF, EGF, PDGF, celiprolol, activins, connective tissue growth factors, MMP inhibitors, cathepsin inhibitors, interleukin inhibitors, TRAIL, VEGF inhibitors, and CD binding agents. In some instances, a therapeutic agent can be an antibody such as, but not limited to, Avastin, Eylea, Humira, ReoPro, Campath, tocilizumab, Ilaris, Removab, Cimzia, Erbitux, Zenapax, Prolia, Raptiva, Rexomun, Abegrin, HuZAF, Simponi, Igovomab, IMAB362, Imciromab, Remicade, Yervoy, Tysabri, Theracim, OvaRex, Vectibix, Theragyn, Omnitarg, Cyramza, Lucentis, Antova, Actemra, Herceptin, Ektomab, Stelara, HumaSPECT, HuMax-EGFr, HuMax-CD4. A therapeutic agent can target tumors, arthritis, or osteoporosis.

Disclosed are nanoparticles comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the spacer moiety can be located between the active agent and the peptide. In some instances, the spacer moiety can comprise aminohexanoic acid. In some instances, the spacer moiety can be one or more glycines or PEG.

Disclosed are nanoparticles comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the peptides can have a higher affinity to degraded collagen than a conventional collagen hybridizing peptide. In some instances, the peptides do not bind native collagen.

Disclosed are nanoparticles comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the N-terminal amino acid comprises an acetyl group. In some instances, the C-terminal amino acid comprises an amino group.

### F. Methods of Detecting

Disclosed are methods of detecting denatured or damaged collagen comprising administering to a subject having a disease or injury involving collagen damage a pharmaceutically acceptable amount of one or more of the disclosed compositions herein.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉is aza-glycine, wherein n¹ and n² can be an integer from 1 to 20.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide , wherein the peptides comprising the sequence (X₁₋Y₁₋Z₁)-(X₂₋Y₂₋Z₂)-(Xₙ₋Yₙ₋Zₙ) (SEQ ID NO:1-39, 82), wherein n can be from 1-41, and X, Y, and Z are glycine, proline, a modified proline or aza-glycine, and at least one of the amino acids in the sequence is aza-glycine. Disclosed are peptides that comprise between 1 and 41 amino acid triplets of the sequence X-Y-Z, wherein X, Y, and Z can be glycine, proline, a modified proline or aza-glycine and each triplet can have the same or different sequence. In some instances, the X, Y, and Z in each triplet can be the same as the X, Y, and Z in every triplet, respectively. In some instances, the X, Y, and Z in each triplet can be different from one or more of the X, Y, and Z′s, respectively, in the other triplets.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉is aza-glycine, wherein n¹ and n² can be an integer from 1 to 20, wherein no more than one of Xaa₁, Xaa₂, Xaa₃, Xaa₄, Xaa₅, Xaa₆, Xaa₇, Xaa₈, or Xaa₉ is aza-glycine. In some instances, Xaa₁, Xaa₂, and Xaa₃ are not the same amino acid. In some instances, Xaa₄, Xaa₅, and Xaa₆ are not the same amino acid. In some instances, Xaa₇, Xaa₈, and Xaa₉ are not the same amino acid. In some instances, at least two of Xaa₁, Xaa₂, and Xaa₃ are not the same amino acid. In some instances, at least two of Xaa₄, Xaa₅, and Xaa₆ are not the same amino acid. In some instances, at least two of Xaa₇, Xaa₈, and Xaa₉ are not the same amino acid.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉is aza-glycine, wherein the peptides comprises the sequence: (Gly-Pro-Hyp)₃-azGly-Pro-Hyp-(Gly-Pro-Hyp)₃ (SEQ ID NO:79); (Pro-Hyp-Gly)₃-Pro-Hyp-azGly-(Pro-Hyp-Gly)₃ (SEQ ID NO:80); or (Pro-Hyp-Gly)₃-Pro-Pro-azGly-(Pro-Hyp-Gly)₃ (SEQ ID NO:81).

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉is aza-glycine, wherein n¹ and n² can be an integer from 1 to 20, wherein the active agent can be a detectable moiety or a therapeutic agent. In some instances, wherein the detectable moiety is a fluorescent dye, radioactive isotope, magnetic bead, metallic bead, colloidal particle, near-infra red dye, or an electron-dense reagents. In some instances, the detectable moiety can be attached to the N-terminal portion of the peptide. Thus, detectable moieties can be, but are not limited to, fluorescent moieties, radioactive moieties, electronic moieties, and indirect moieties such as biotin or digoxigenin. When indirect moieties are used, a secondary binding agent that binds the indirect moiety can be used to detect the presence of a bound collagen hybridizing peptide. These secondary binding agents can comprise antibodies, haptens, or other binding partners (e.g., avidin) that bind to the indirect moieties. In some instances, a detectable moiety can be attached to the N-terminal, C-terminal, or both portions of the peptide. In some instances, the therapeutic agent can be a therapeutic known to treat a disease or injury involving collagen damage. For example, the therapeutic agent can be, but is not limited to, any suitable pharmaceutical or other therapeutic agent, including but not limited to, osteogenic promoters, antimicrobials, anti-inflammatory agents, polypeptides such as recombinant proteins, cytokines or antibodies, small molecule chemicals or any combination thereof. In some instances, a therapeutic agent can be a cancer drug, arthritis drug or osteoporosis drug. Therapeutic agents can be capable of promoting bone growth, decreasing inflammation, promoting collagen stability. Examples of therapeutic agents can include, but is not limited to, bone morphogenic protein (BMP), G-CSF, FGF, BMP-2, BMP-3, FGF-2, FGF-4, anti-sclerostin antibody, growth hormone, IGF-1, VEGF, TGF-β, KGF, FGF-10, TGF-α, TGF-β1, TGF-β receptor, CT, GH, GM-CSF, EGF, PDGF, celiprolol, activins, connective tissue growth factors, MMP inhibitors, cathepsin inhibitors, interleukin inhibitors, TRAIL, VEGF inhibitors, and CD binding agents. In some instances, a therapeutic agent can be an antibody such as, but not limited to, Avastin, Eylea, Humira, ReoPro, Campath, tocilizumab, Ilaris, Removab, Cimzia, Erbitux, Zenapax, Prolia, Raptiva, Rexomun, Abegrin, HuZAF, Simponi, Igovomab, IMAB362, Imciromab, Remicade, Yervoy, Tysabri, Theracim, OvaRex, Vectibix, Theragyn, Omnitarg, Cyramza, Lucentis, Antova, Actemra, Herceptin, Ektomab, Stelara, HumaSPECT, HuMax-EGFr, HuMax-CD4. A therapeutic agent can target tumors, arthritis, or osteoporosis.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉is aza-glycine, wherein n¹ and n² can be an integer from 1 to 20, wherein the spacer moiety can be located between the active agent and the peptide. In some instances, the spacer moiety can comprise aminohexanoic acid. In some instances, the spacer moiety can be one or more glycines or PEG.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉is aza-glycine, wherein n¹ and n² can be an integer from 1 to 20, wherein the composition further comprises a nanoparticle. For example, the disclosed methods of detecting can be performed by administering any of the disclosed nanoparticles herein.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉is aza-glycine, wherein n¹ and n² can be an integer from 1 to 20, wherein the peptides can have a higher affinity to degraded collagen than a conventional collagen hybridizing peptide. In some instances, the peptides do not bind native collagen.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉is aza-glycine, wherein n¹ and n² can be an integer from 1 to 20, wherein the N-terminal amino acid comprises an acetyl group. In some instances, the C-terminal amino acid comprises an amino group.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉is aza-glycine, wherein n¹ and n² can be an integer from 1 to 20, further comprising a step of heating the composition prior to administration. In some instances, heating comprises incubating the composition at a temperature at least one degree higher than the melting transition temperature of the peptide. In some instances, heating comprises incubating the composition at a temperature of at least 50-95° C. In some instances, heating can be necessary to prevent trimers of the disclosed peptides (i.e. collagen hybridizing peptides). In some instances, peptides heated to such temperatures can be quickly cooled to room temperature or body temperature immediately prior to administration.

### G. Methods of Treating

Disclosed are methods of treating a disease or injury involving collagen damage comprising administering to a subject having a disease or injury involving collagen damage with any of the disclosed compositions herein.

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20.

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (X1-Y1-Z1)-(X2-Y2-Z2)-(Xn-Yn-Zn) (SEQ ID NOs:1-39, 82), wherein n can be from 1-41, and X, Y, and Z are glycine, proline, a modified proline and/or aza-glycine, and at least one of the amino acids in the sequence is aza-glycine.

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein no more than one of Xaa₁, Xaa₂, Xaa₃, Xaa₄, Xaa₅, Xaa₆, Xaa₇, Xaa₈, or Xaa₉is aza-glycine. In some instances, Xaa₁, Xaa₂, and Xaa₃ are not the same amino acid. In some instances, Xaa₄, Xaa₅, and Xaa₆ are not the same amino acid. In some instances, Xaa₇, Xaa₈, and Xaa₉ are not the same amino acid. In some instances, at least two of Xaa₁, Xaa₂, and Xaa₃ are not the same amino acid. In some instances, at least two of Xaa₄, Xaa₅, and Xaa₆ are not the same amino acid. In some instances, at least two of Xaa₇, Xaa₈, and Xaa₉ are not the same amino acid.

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein the peptides comprises the sequence: (Gly-Pro-Hyp)₃-azGly-Pro-Hyp-(Gly-Pro-Hyp)₃ (SEQ ID NO:79); (Pro-Hyp-Gly)₃-Pro-Hyp-azGly-(Pro-Hyp-Gly)₃ (SEQ ID NO:80); or (Pro-Hyp-Gly)₃-Pro-Pro-azGly-(Pro-Hyp-Gly)₃ (SEQ ID NO:81).

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the active agent can be a detectable moiety or a therapeutic agent. In some instances, wherein the detectable moiety is a fluorescent dye, radioactive isotope, magnetic bead, metallic bead, colloidal particle, near-infra red dye, or an electron-dense reagents. In some instances, the detectable moiety can be attached to the N-terminal portion of the peptide. Thus, detectable moieties can be, but are not limited to, fluorescent moieties, radioactive moieties, electronic moieties, and indirect moieties such as biotin or digoxigenin. When indirect moieties are used, a secondary binding agent that binds the indirect moiety can be used to detect the presence of a bound collagen hybridizing peptide. These secondary binding agents can comprise antibodies, haptens, or other binding partners (e.g., avidin) that bind to the indirect moieties. In some instances, a detectable moiety can be attached to the N-terminal, C-terminal, or both portions of the peptide. In some instances, the therapeutic agent can be a therapeutic known to treat a disease or injury involving collagen damage. For example, the therapeutic agent can be, but is not limited to, any suitable pharmaceutical or other therapeutic agent, including but not limited to, osteogenic promoters, antimicrobials, anti-inflammatory agents, polypeptides such as recombinant proteins, cytokines or antibodies, small molecule chemicals or any combination thereof. In some instances, a therapeutic agent can be a cancer drug, arthritis drug or osteoporosis drug. Therapeutic agents can be capable of promoting bone growth, decreasing inflammation, promoting collagen stability. Examples of therapeutic agents can include, but is not limited to, bone morphogenic protein (BMP), G-CSF, FGF, BMP-2, BMP-3, FGF-2, FGF-4, anti-sclerostin antibody, growth hormone, IGF-1, VEGF, TGF-β, KGF, FGF-10, TGF-α, TGF-β1, TGF-β receptor, CT, GH, GM-CSF, EGF, PDGF, celiprolol, activins, connective tissue growth factors, MMP inhibitors, cathepsin inhibitors, interleukin inhibitors, TRAIL, VEGF inhibitors, and CD binding agents. In some instances, a therapeutic agent can be an antibody such as, but not limited to, Avastin, Eylea, Humira, ReoPro, Campath, tocilizumab, Ilaris, Removab, Cimzia, Erbitux, Zenapax, Prolia, Raptiva, Rexomun, Abegrin, HuZAF, Simponi, Igovomab, IMAB362, Imciromab, Remicade, Yervoy, Tysabri, Theracim, OvaRex, Vectibix, Theragyn, Omnitarg, Cyramza, Lucentis, Antova, Actemra, Herceptin, Ektomab, Stelara, HumaSPECT, HuMax-EGFr, HuMax-CD4. A therapeutic agent can target tumors, arthritis, or osteoporosis.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉is aza-glycine, wherein n¹ and n² can be an integer from 1 to 20, wherein the spacer moiety can be located between the active agent and the peptide. In some instances, the spacer moiety can comprise aminohexanoic acid. In some instances, the spacer moiety can be one or more glycines or PEG.

Disclosed are methods of detecting denatured or damaged collagen comprising administering to a subject having a disease or injury involving collagen damage a pharmaceutically acceptable amount of a composition comprising a peptide conjugate, wherein the peptide conjugate comprises an active agent, a spacer moiety, and a peptide wherein the peptide comprises the sequence (X1-Y1-Z1)-(X2-Y2-Z2)-(Xn-Yn-Zn) (SEQ ID NOs:1-39, 82), wherein n can be from 1-41, and X, Y, and Z are glycine, proline, a modified proline and/or aza-glycine, and at least one of the amino acids in the sequence is aza-glycine.

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the composition further comprises a nanoparticle. For example, the disclosed methods of detecting can be performed by administering any of the disclosed nanoparticles herein.

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaaₛ-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the peptides can have a higher affinity to degraded collagen than a conventional collagen hybridizing peptide. In some instances, the peptides do not bind native collagen.

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, wherein the N-terminal amino acid comprises an acetyl group. In some instances, the C-terminal amino acid comprises an amino group.

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20, further comprising a step of heating the composition prior to administration. In some instances, heating comprises incubating the composition at a temperature at least one degree higher than the melting transition temperature of the peptide. In some instances, heating comprises incubating the composition at a temperature of at least 50-95° C. In some instances, heating can be necessary to prevent trimers of the disclosed peptides (i.e. collagen hybridizing peptides). In some instances, peptides heated to such temperatures can be quickly cooled to room temperature or body temperature immediately prior to administration.

Disclosed are methods of treating a disease or injury involving collagen damage comprising administering to a subject having a disease or injury involving collagen damage a peptide conjugate, wherein the peptide conjugate comprises an active agent, a spacer moiety, and a peptide wherein the peptide comprises the sequence (X₁₋Y₁₋Z₁)-(X₂₋Y₂₋Z₂)-(Xₙ₋Yₙ₋Zₙ) (SEQ ID NO:1-39, 82), wherein n can be from 1-41, and X, Y, and Z are glycine, proline, a modified proline or aza-glycine, and at least one of the amino acids in the sequence is aza-glycine. Disclosed are peptides that comprise between 1 and 41 amino acid triplets of the sequence X-Y-Z, wherein X, Y, and Z can be glycine, proline, a modified proline or aza-glycine and each triplet can have the same or different sequence. In some instances, the X, Y, and Z in each triplet can be the same as the X, Y, and Z in every triplet, respectively. In some instances, the X, Y, and Z in each triplet can be different from one or more of the X, Y, and Z's, respectively, in the other triplets.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising any one or more of the disclosed peptide conjugates, nanoparticles or compositions. The kits also can contain a solid support.

In some instances, disclosed are kits comprising a peptide conjugate, wherein the peptide conjugate comprises an active agent, a spacer moiety, and a peptide, wherein the peptide comprises the sequence (Xaa₁-Xaa₂-Xaa₃)ₙ¹-Xaa₄-Xaa₅-Xaa₆-(Xaa₇-Xaa₈-Xaa₉)ₙ² (SEQ ID NOs:1-39), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ and n² can be an integer from 1 to 20. In some instances, the peptide can be attached to or immobilized on a solid support.

In some instances, disclosed are kits comprising a peptide conjugate, wherein the peptide conjugate comprises an active agent, a spacer moiety, and a peptide, wherein the peptide comprises the sequence (X₁₋Y₁₋Z₁)-(X₂₋Y₂₋Z₂)-(Xₙ₋Yₙ₋Zₙ) (SEQ ID NO:1-39, 82), wherein n can be from 1-41, and X, Y, and Z are glycine, proline, a modified proline or aza-glycine, and at least one of the amino acids in the sequence is aza-glycine.

Also disclosed are kits comprising one or more of the disclosed peptides. In some instances, the peptide can be attached to or immobilized on a solid support.

### EXAMPLES

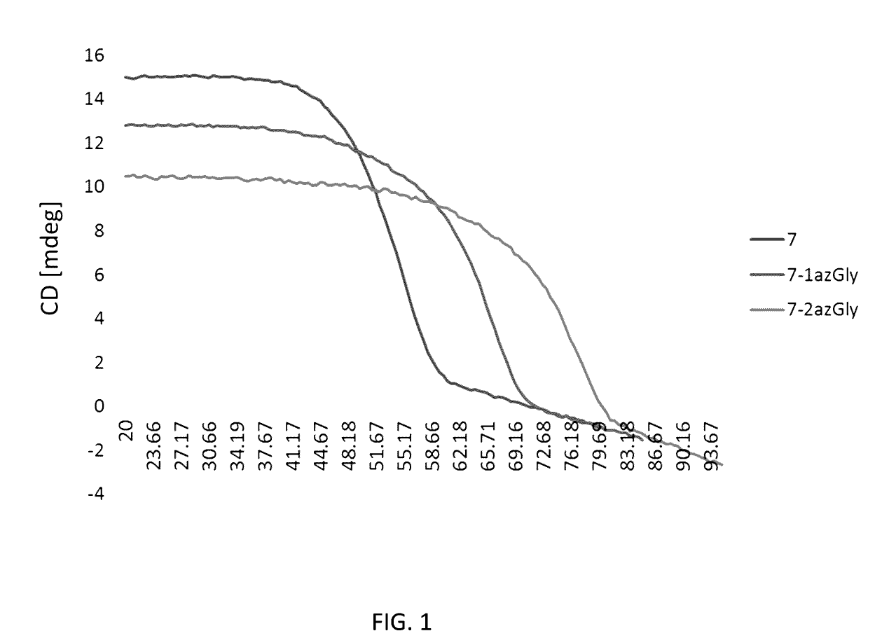

The sigmoidal shape of the melting curve indicates that the compound Ac(GPO)₃AzGPO(GPO)₃ and Ac(GPO)₂AzGPOGPOAzGPO(GPO)₂ has triple helical structure (FIG. 1).

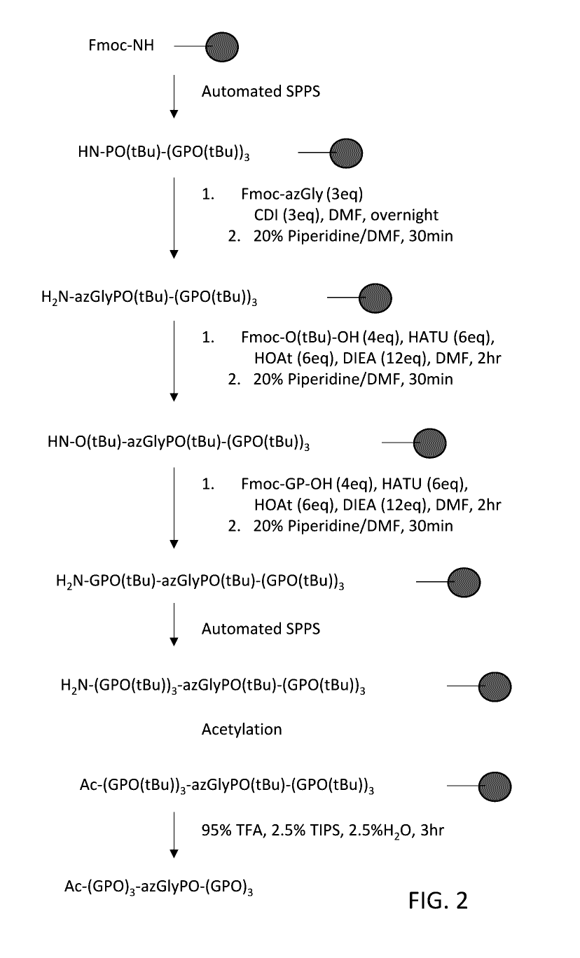

The synthetic scheme of Ac-(GPO)3-azGPO-(GPO)3 (FIG. 2) is provided by the detailed steps for coupling Fmoc-NH-NH2: 1. Weigh CDI into the vial; 2. Dissolve in DMF; 3. Add Fmoc-NH-NH2; 4. Mix for 5 min; 5. Add mixture to the resin; and 6. Mix for 24 hr.

The synthesis of Ac-(GPO)3-azGPO-(GPO)3 involves using Solid Phase Peptide Synthesis. PO-(GPO)3 was synthesized on a tentagel resin using a peptide synthesizer. Coupling of the amino acid to the resin was started from the amino acid on the C-terminal of the sequence, in this case is O. Coupling of each amino acid was achieved by utilizing C1-HOBT (4eq), HBTU (4eq), DIEA (6eq) in DMF. The reaction was run for 1 hr for Gly coupling or 2 hr for Pro or Hyp coupling. Subsequent amine deprotection after amino acid coupling was performed in 20% piperidine in DMF. The reaction was run for 30 min. Upon completion of PO-GPO-GPO-GPO sequence, azaG was coupled utilizing CDI (3eq), Fmoc-NH-NH2 (3eq) in DMF. The reaction was run for overnight (12 hr). Then, amine deprotection was performed using 20% piperidine in DMF. Coupling of Hyp after the azaG was accomplished was performed using HATU (6eq), HOAt (6eq), DIEA (12eq) in DMF. Then, the amine was deprotected using 20% piperidine in DMF. Coupling of GlyPro after Hyp was accomplished using HATU (6eq), HOAt (6eq), DIEA (12eq) in DMF. Then, the amine was protected using 20% piperidine in DMF.

Using Solid Phase Peptide Synthesis, the peptide was extended to (GPO)3-azGPO-(GPO)3. Acetylation of (GPO)3-azGPO-(GPO)3 is achieved using capping mix comprising of 5 mL DMF, 0.2375 mL acetic anhydride, 112.5 μL DIEA, 10.1 mg HOAt. 3 mL capping mix was diluted with 1.5 mL DMF and used to acetylate 0.05 mmol resin. The reaction was run for 1 hr. Cleaving the peptide from the resin is performed using 95% TFA, 2.5% TIPS, 2.5% H2O. (Cleaving volume is based on 20 mL/gr dry weight of the resin)

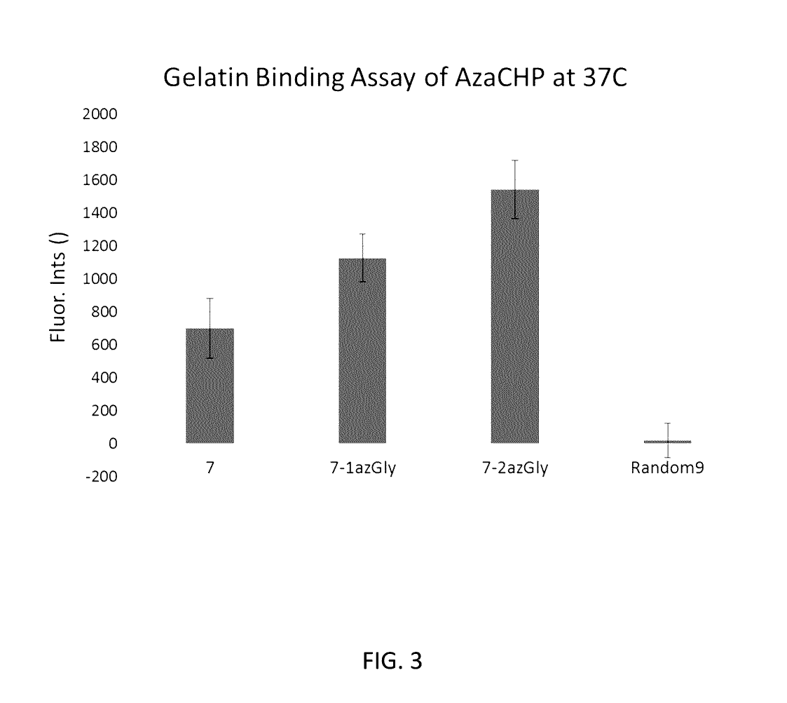

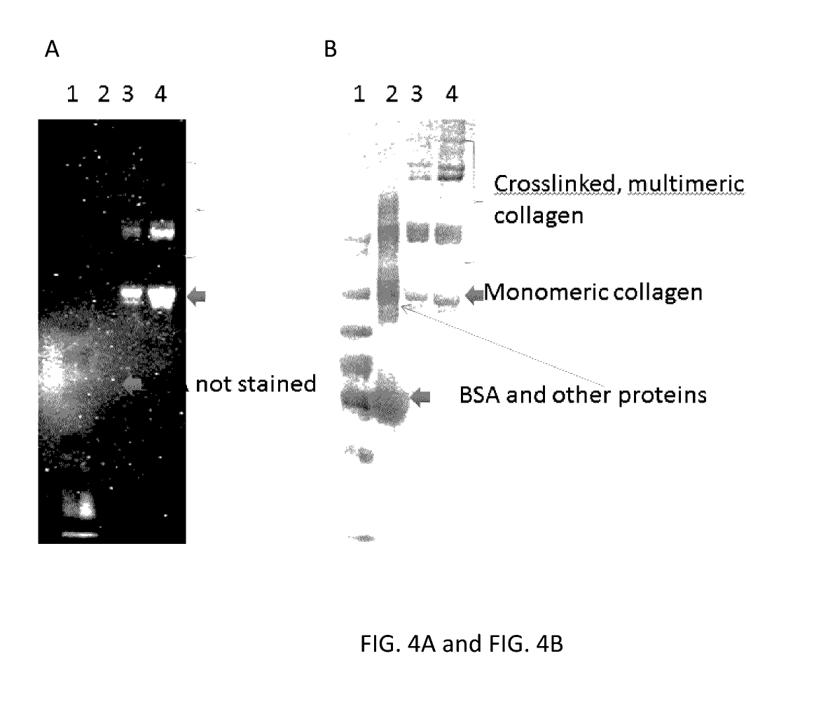

FIG. 3 indicates that AzGPO has higher binding affinity to gelatin than conventional GPO. Binding of azGPO7 is much higher than GPO7. Replacement of one G with one azG within azGPO7 as exemplified by (GPO)2AzGPOGPOAzGPO(GPO)2 further increases the binding affinity to gelatin. FIG. 4 shows that Aza-CHP binds to denatured collagen, but it does not bind to denatured BSA. This result shows specificity of aza-CHP for hybridizing with collagen strand.

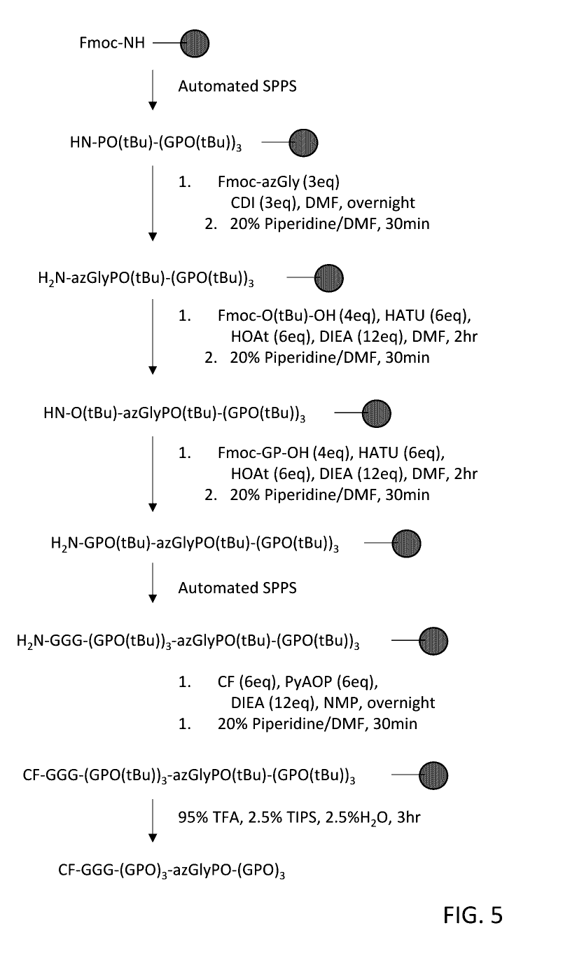

The synthetic scheme of CF-G3-(GPO)₃-azGPO-(GPO)₃ (FIG. 5) is provided by the detailed steps for coupling Fmoc-NH-NH₂: 1. Weigh CDI into the vial; 2. Dissolve in DMF; 3. Add Fmoc-NH-NH₂; 4. Mix for 5 min; 5. Add mixture to the resin; and 6. Mix for 24 hr.

The synthesis of CF-G₃-(GPO)₃-azGPO-(GPO)₃ involves using Solid Phase Peptide Synthesis. PO-(GPO)₃ was synthesized on a tentagel resin using a peptide synthesizer. Coupling of the amino acid to the resin was started from the amino acid on the C-terminal of the sequence, in this case is O. Coupling of each amino acid was achieved by utilizing C1-HOBT (4eq), HBTU (4eq), DIEA (6eq) in DMF. The reaction was run for 1 hr for Gly coupling or 2 hr for Pro or Hyp coupling. Subsequent amine deprotection after amino acid coupling was performed in 20% piperidine in DMF. The reaction was run for 30 min. Upon completion of PO-GPO-GPO-GPO sequence, azaG was coupled utilizing CDI (3eq), Fmoc-NH-NH₂ (3eq) in DMF. The reaction was run for overnight (12 hr). Then, amine deprotection was performed using 20% piperidine in DMF. Coupling of Hyp after the azaG was accomplished was performed using HATU (6eq), HOAt (6eq), DIEA (12eq) in DMF. Then, the amine was deprotected using 20% piperidine in DMF. Coupling of GlyPro after Hyp was accomplished using HATU (6eq), HOAt (6eq), DIEA (12eq) in DMF. Then, the amine was protected using 20% piperidine in DMF.

Using Solid Phase Peptide Synthesis, the peptide was extended to GGG-(GPO)₃-azGPO-(GPO)₃. CF coupling to GGG-(GPO)₃-azGPO-(GPO)₃ is achieved using PyAOP (6eq), DIEA (12eq) in NMP. Cleaving the peptide from the resin is performed using 95% TFA, 2.5% TIPS, 2.5% H₂O. (Cleaving volume is based on 20 mL/gr dry weight of the resin)

Those skilled in the art will recognize, or be able to ascertain using no more than routine experimentation, many equivalents to the specific embodiments of the method and compositions described herein. Such equivalents are intended to be encompassed by the following claims.

1-145. (canceled)

146. A peptide conjugate comprising an active agent, a spacer moiety, and a peptide, wherein the peptide comprises the sequence (X₁-Y₁-Z₁)-(X₂-Y₂-Z₂)-(Xₙ₋Yₙ-Zₙ) (SEQ ID NO:1-39, 82), wherein n=1-41, and X, Y, and Z are glycine, proline, a modified proline and/or aza-glycine, and at least one of the amino acids in the sequence is aza-glycine.

147. The peptide conjugate of claim 146, wherein the active agent is a detectable moiety or a therapeutic agent.

148. The peptide conjugate of claim 147, wherein the detectable moiety is a fluorescent dye, radioactive isotope, contrast agent, magnetic bead, metallic bead, colloidal particle, near-infra red dye, or an electron-dense reagents.

149. The peptide conjugate of claim 147, wherein the detectable moiety is attached to the N-terminal portion of the peptide.

150. The peptide conjugate of claim 146, wherein the spacer moiety is located between the active agent and the peptide.

151. The peptide conjugate of claim 146, wherein the spacer moiety comprises aminohexanoic acid or one or more glycines.

152. The peptide conjugate of claim 147, wherein the therapeutic agent is a therapeutic known to treat a disease or injury involving collagen damage.

153. The peptide conjugate of claim 146, wherein the peptides have a higher affinity to degraded collagen than a conventional collagen hybridizing peptide.

154. The peptide conjugate of claim 146, wherein the peptides do not bind native collagen.

155. A composition comprising the peptide conjugate of claim 146.

156. The composition of claim 155, wherein the active agent is a detectable moiety or a therapeutic agent.

157. The composition of claim 156, wherein the detectable moiety is a fluorescent dye, radioactive isotope, contrast agent, magnetic bead, metallic bead, colloidal particle, near-infra red dye, or an electron-dense reagents.

158. The composition of claim 155, wherein the spacer moiety is located between the active agent and the peptide.

159. The composition of claim 156, wherein the therapeutic agent is a therapeutic known to treat a disease or injury involving collagen damage.

160. A method of detecting denatured collagen comprising administering to a subject having a disease or injury involving collagen damage a pharmaceutically acceptable amount of a composition comprising the peptide conjugate of claim 146.

161. The method of claim 160, wherein the active agent is a detectable moiety

162. The method of claim 161, wherein the detectable moiety is a fluorescent dye, radioactive isotope, contrast agent, magnetic bead, metallic bead, colloidal particle, near-infra red dye, or an electron-dense reagents.

163. A method of treating a disease or injury involving collagen damage comprising administering to a subject having a disease or injury involving collagen damage the peptide conjugate of claim 146.

164. The method of claim 163, wherein the active agent is a therapeutic agent.

165. The method of claim 164, wherein the therapeutic agent is a therapeutic known to treat a disease or injury involving collagen damage.

166. A peptide comprising the sequence (X₁-Y₁-Z₁)-(X₂-Y₂-Z₂)-(Xₙ₋Yₙ-Zₙ) (SEQ ID NO:1-39, 82), wherein n=1-41, and X, Y, and Z are glycine, proline, a modified proline and/or aza-glycine, and at least one of the amino acids in the sequence is aza-glycine.


SEQUENCE LISTING (96 sequences)

SEQ ID NO: 1 - protein, 9 aa, Artificial Sequence
  synthetic construct; collagen hybridizing peptide
XXXXXXXXX

SEQ ID NO: 2 - protein, 12 aa, Artificial Sequence
  synthetic construct; collagen hybridizing peptide
XXXXXXXXXXXX

SEQ ID NO: 3 - protein, 15 aa, Artificial Sequence
  synthetic construct; collagen hybridizing peptide
XXXXXXXXXXXXXXX

SEQ ID NO: 4 - protein, 18 aa, Artificial Sequence
  synthetic construct; collagen hybridizing peptide
XXXXXXXXXXXXXXXXXX

SEQ ID NO: 5 - protein, 21 aa, Artificial Sequence
  synthetic construct; collagen hybridizing peptide
XXXXXXXXXXXXXXXXXXXXX

SEQ ID NO: 6 - protein, 24 aa, Artificial Sequence
  synthetic construct; collagen hybridizing peptide
XXXXXXXXXXXXXXXXXXXXXXXX

SEQ ID NO: 7 - protein, 27 aa, Artificial Sequence
  synthetic construct; collagen hybridizing peptide
XXXXXXXXXXXXXXXXXXXXXXXXXXX

SEQ ID NO: 8 - protein, 30 aa, Artificial Sequence
  synthetic construct; collagen hybridizing peptide
XXXXXXXXXXXXXXXXXXXXXXXXXXXXXX

SEQ ID NO: 9 - protein, 33 aa, Artificial Sequence
  synthetic construct; collagen hybridizing peptide
XXXXXXXXXXXXXXXXXXXXXXXXXXXXXXXXX

SEQ ID NO: 10 - protein, 36 aa, Artificial Sequence
  synthetic construct; collagen hybridizing peptide
XXXXXXXXXXXXXXXXXXXXXXXXXXXXXXXXXXXX

SEQ ID NO: 11 - protein, 39 aa, Artificial Sequence
  synthetic construct; collagen hybridizing peptide
XXXXXXXXXXXXXXXXXXXXXXXXXXXXXXXXXXXXXXX

SEQ ID NO: 12 - protein, 42 aa, Artificial Sequence
  synthetic construct; collagen hybridizing peptide
XXXXXXXXXXXXXXXXXXXXXXXXXXXXXXXXXXXXXXXXXX

SEQ ID NO: 13 - protein, 45 aa, Artificial Sequence
  synthetic construct; collagen hybridizing peptide
XXXXXXXXXXXXXXXXXXXXXXXXXXXXXXXXXXXXXXXXXXXXX

SEQ ID NO: 14 - protein, 48 aa, Artificial Sequence
  synthetic construct; collagen hybridizing peptide
XXXXXXXXXXXXXXXXXXXXXXXXXXXXXXXXXXXXXXXXXXXXXXXX

SEQ ID NO: 15 - protein, 51 aa, Artificial Sequence
  synthetic construct; collagen hybridizing peptide
XXXXXXXXXXXXXXXXXXXXXXXXXXXXXXXXXXXXXXXXXXXXXXXXXXX

SEQ ID NO: 16 - protein, 54 aa, Artificial Sequence
  synthetic construct; collagen hybridizing peptide
XXXXXXXXXXXXXXXXXXXXXXXXXXXXXXXXXXXXXXXXXXXXXXXXXXXXXX

SEQ ID NO: 17 - protein, 57 aa, Artificial Sequence
  synthetic construct; collagen hybridizing peptide
XXXXXXXXXXXXXXXXXXXXXXXXXXXXXXXXXXXXXXXXXXXXXXXXXXXXXXXXX

SEQ ID NO: 18 - protein, 60 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXX

SEQ ID NO: 19 - protein, 63 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXX

SEQ ID NO: 20 - protein, 66 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXX

SEQ ID NO: 21 - protein, 69 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXX

SEQ ID NO: 22 - protein, 72 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXX

SEQ ID NO: 23 - protein, 75 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXX

SEQ ID NO: 24 - protein, 78 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXX

SEQ ID NO: 25 - protein, 81 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 26 - protein, 84 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 27 - protein, 87 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 28 - protein, 90 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 29 - protein, 93 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 30 - protein, 96 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 31 - protein, 99 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 32 - protein, 102 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 33 - protein, 105 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 34 - protein, 108 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 35 - protein, 111 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 36 - protein, 114 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 37 - protein, 117 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 38 - protein, 120 aa, Artificial Sequence
  ynthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 39 - protein, 123 aa, Artificial Sequence
  synthetic construct; collagen hybridizing peptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 40 - protein, 9 aa, Artificial Sequence
  synthetic construct; collagen hybridizing peptide
GPPGPPGPP

SEQ ID NO: 41 - protein, 12 aa, Artificial Sequence
  synthetic construct; collagen hybridizing peptide
GPPGPPGPPGPP

SEQ ID NO: 42 - protein, 15 aa, Artificial Sequence
  synthetic construct; collagen hybridizing peptide
GPPGPPGPPGPPGPP

SEQ ID NO: 43 - protein, 18 aa, Artificial Sequence
  synthetic construct; collagen hybridizing peptide
GPPGPPGPPGPPGPPGPP

SEQ ID NO: 44 - protein, 21 aa, Artificial Sequence
  synthetic construct; collagen hybridizing peptide
GPPGPPGPPGPPGPPGPPGPP

SEQ ID NO: 45 - protein, 24 aa, Artificial Sequence
  synthetic construct; collagen hybridizing peptide
GPPGPPGPPGPPGPPGPPGPPGPP

SEQ ID NO: 46 - protein, 27 aa, Artificial Sequence
  synthetic construct; collagen hybridizing peptide
GPPGPPGPPGPPGPPGPPGPPGPPGPP

SEQ ID NO: 47 - protein, 30 aa, Artificial Sequence
  synthetic construct; collagen hybridizing peptide
GPPGPPGPPGPPGPPGPPGPPGPPGPPGPP

SEQ ID NO: 48 - protein, 33 aa, Artificial Sequence
  synthetic construct; collagen hybridizing peptide
GPPGPPGPPGPPGPPGPPGPPGPPGPPGPPGPP

SEQ ID NO: 49 - protein, 36 aa, Artificial Sequence
  synthetic construct; collagen hybridizing peptide
GPPGPPGPPGPPGPPGPPGPPGPPGPPGPPGPPGPP

SEQ ID NO: 50 - protein, 39 aa, Artificial Sequence
  synthetic construct; collagen hybridizing peptide
GPPGPPGPPGPPGPPGPPGPPGPPGPPGPPGPPGPPGPP

SEQ ID NO: 51 - protein, 42 aa, Artificial Sequence
  synthetic construct; collagen hybridizing peptide
GPPGPPGPPGPPGPPGPPGPPGPPGPPGPPGPPGPPGPPGPP

SEQ ID NO: 52 - protein, 45 aa, Artificial Sequence
  synthetic construct; collagen hybridizing peptide
GPPGPPGPPGPPGPPGPPGPPGPPGPPGPPGPPGPPGPPGPPGPP

SEQ ID NO: 53 - protein, 48 aa, Artificial Sequence
  synthetic construct; collagen hybridizing peptide
GPPGPPGPPGPPGPPGPPGPPGPPGPPGPPGPPGPPGPPGPPGPPGPP

SEQ ID NO: 54 - protein, 51 aa, Artificial Sequence
  synthetic construct; collagen hybridizing peptide
GPPGPPGPPGPPGPPGPPGPPGPPGPPGPPGPPGPPGPPGPPGPPGPPGPP

SEQ ID NO: 55 - protein, 54 aa, Artificial Sequence
  synthetic construct; collagen hybridizing peptide
GPPGPPGPPGPPGPPGPPGPPGPPGPPGPPGPPGPPGPPGPPGPPGPPGPPGPP

SEQ ID NO: 56 - protein, 57 aa, Artificial Sequence
  synthetic construct; collagen hybridizing peptide
GPPGPPGPPGPPGPPGPPGPPGPPGPPGPPGPPGPPGPPGPPGPPGPPGPPGPPGPP

SEQ ID NO: 57 - protein, 60 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPP

SEQ ID NO: 58 - protein, 63 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPP

SEQ ID NO: 59 - protein, 66 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPP

SEQ ID NO: 60 - protein, 69 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPP

SEQ ID NO: 61 - protein, 72 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPP

SEQ ID NO: 62 - protein, 75 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPP

SEQ ID NO: 63 - protein, 78 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPP

SEQ ID NO: 64 - protein, 81 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 65 - protein, 84 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 66 - protein, 87 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 67 - protein, 90 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 68 - protein, 93 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 69 - protein, 96 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 70 - protein, 99 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 71 - protein, 102 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 72 - protein, 105 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 73 - protein, 108 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 74 - protein, 111 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 75 - protein, 114 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 76 - protein, 117 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 77 - protein, 120 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 78 - protein, 123 aa, Artificial Sequence
  synthetic construct; collagen hybridizing peptide
GPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPP

SEQ ID NO: 79 - protein, 21 aa, Artificial Sequence
  synthetic construct; collagen hybridizing peptide
GPPGPPGPPGPPGPPGPPGPP

SEQ ID NO: 80 - protein, 21 aa, Artificial Sequence
  synthetic construct; collagen hybridizing peptide
PPGPPGPPGPPGPPGPPGPPG

SEQ ID NO: 81 - protein, 21 aa, Artificial Sequence
  synthetic construct; collagen hybridizing peptide
PPGPPGPPGPPGPPGPPGPPG

SEQ ID NO: 82 - protein, 6 aa, Artificial Sequence
  synthetic construct; collagen hybridizing peptide
XXXXXX

SEQ ID NO: 83 - protein, 21 aa, Artificial Sequence
  synthetic construct; collagen hybridizing peptide
GPPGPPGPPGPPGPPGPPGPP

SEQ ID NO: 84 - protein, 21 aa, Artificial Sequence
  synthetic construct; collagen hybridizing peptide
GPPGPPGPPGPPGPPGPPGPP

SEQ ID NO: 85 - protein, 21 aa, Artificial Sequence
  synthetic construct; collagen hybridizing peptide
GPPGPPGPPGPPGPPGPPGPP

SEQ ID NO: 86 - protein, 21 aa, Artificial Sequence
  synthetic construct; collagen hybridizing peptide
GPPGPPGPPGPPGPPGPPGPP

SEQ ID NO: 87 - protein, 21 aa, Artificial Sequence
  synthetic construct; collagen hybridizing peptide
GPPGPPGPPGPPGPPGPPGPP

SEQ ID NO: 88 - protein, 21 aa, Artificial Sequence
  synthetic construct; collagen hybridizing peptide
GPPGPPGPPGPPGPPGPPGPP

SEQ ID NO: 89 - protein, 21 aa, Artificial Sequence
  synthetic construct; collagen hybridizing peptide
GPPGPPGPPGPPGPPGPPGPP

SEQ ID NO: 90 - protein, 21 aa, Artificial Sequence
  synthetic construct; collagen hybridizing peptide
GPPGPPGPPGPPGPPGPPGPP

SEQ ID NO: 91 - protein, 21 aa, Artificial Sequence
  synthetic construct; collagen hybridizing peptide
GPPGPPGPPGPPGPPGPPGPP

SEQ ID NO: 92 - protein, 21 aa, Artificial Sequence
  synthetic construct; collagen hybridizing peptide
GPPGPPGPPGPPGPPGPPGPP

SEQ ID NO: 93 - protein, 21 aa, Artificial Sequence
  synthetic construct; collagen hybridizing peptide
GPPGPPGPPGPPGPPGPPGPP

SEQ ID NO: 94 - protein, 21 aa, Artificial Sequence
  synthetic construct; artificial sequence
GPPGPPGPPGPPGPPGPPGPP

SEQ ID NO: 95 - protein, 21 aa, Artificial Sequence
  synthetic construct; collagen hybridizing peptide
GPPGPPGPPGPPGPPGPPGPP

SEQ ID NO: 96 - protein, 21 aa, Artificial Sequence
  synthetic construct; collagen hybridizing peptide
GPPGPPGPPGPPGPPGPPGPP